(19) United States
(12) Patent Application Publication
(10) Pub. No.: US 2005/0169978 A1
(43) Pub. Date: Aug. 4, 2005

(54) Wet-micro grinding

(76) Inventors: Shu-Wen Fu, Hsin Chu City (CN); Chien-Hsin D. Cheng, Marietta, GA (US); Jui-Ching Cheng, Tao Yuan City (TW); Yun-Yi Hsiau, Tao Yuan City (TW)

Correspondence Address: FISH & RICHARDSON PC, 225 FRANKLIN ST, BOSTON, MA, 02110, US

(21) Appl. No.: 10/769,118

(22) Filed: Jan. 29, 2004

(51) Int. Cl. A61K 9/127; A61K 9/16; A61K 9/50

(52) U.S. Cl. 424/450; 514/34; 514/283; 514/449

(57) ABSTRACT

This invention relates to a method for preparing a drug-lipid complex that does not have a captured volume or a drug-containing liposome. The method includes dispersing a drug and one or more phospholipids in an aqueous solution to obtain a mixture and grinding the mixture with a mechanic means to obtain a drug-lipid complex that does not have a captured volume or a drug-containing liposome.

### BACKGROUND

Poor bioavailability and high toxicity are significant problems encountered in preparation of pharmaceutical compositions, particularly those containing an active ingredient that is poorly soluble in water.

As one can improve the dissolution rate of a particulate drug by decreasing its particle sizes, there has been extensive research on techniques of preparing pharmaceutical compositions with smaller particle sizes. Such techniques can be applied to prepare drug-associated lipid complexes. According to reports, drug-associated lipid complexes at high drug-lipid ratios are generally equivalent or greater in efficacy than the same drug in its free form, yet have lower toxicity.

However, techniques for preparing such drug-associated lipid complexes tend to be expensive, as they generally require use of organic solvents. It would be desirable to develop a more cost-effective method.

### SUMMARY

This invention is based on the unexpected discovery that drug nanoparticles can be prepared by grinding a drug and one or more phospholipids in an aqueous solution without using any organic solvents.

In one aspect, this invention features a method for preparing a drug-containing liposome or a drug-lipid complex that does not have a captured volume. The method includes dispersing a drug and one or more phospholipids in an aqueous solution to obtain a mixture and grinding the mixture with a mechanic means to obtain a drug-containing liposome or a drug-lipid complex that does not have a captured volume.

The term “drug-containing liposome” refers to a closed lipid bilayer structure in which the drug molecules are either entrapped in the lipid bilayer structure or are encapsulated in the core defined by the lipid bilayer structure. Liposomes can be unilamellar vesicles (i.e., having a single bilayer structure) or multilamellar vesicles (i.e., having an onion-like, multiple bilayer structure). The term “drug-lipid complex that does not have a captured volume” refers to an acylic and open lipid structure (either single layer or bilayer) that entraps the drug molecules in its layer. It can be distinguished from drug-lipid micelles, which typically have a cyclic or closed lipid structure. The term “drug” refers to a biologically active agent, which confers a therapeutic or otherwise beneficial effect to a subject, e.g., to cure, relieve, alter, affect, ameliorate, or prevent a disease in the subject, or to promote the overall health of the subject. The drug can be either water-soluble (i.e., its water solubility is greater than or equal to 10 mg/mL at about 25° C.), or water-insoluble (i.e., its water-solubility is less than 10 mg/mL at about 25° C.). Examples of drugs include, but are not limited to, amphotericin B, doxorubicin, taxol, or irinotecan, as well as their salts and prodrugs. Examples of phospholipids include, but are not limited to, phosphatidylcholine, phosphatidylglycerol, egg phosphatidylcholine, egg phosphatidylglycerol, dimyristoylphosphatidylcholine, dimyristoylphosphatidylglycerol, distearoylphosphatidylcholine, distearoylphosphatidylglycerol, hydrogenated soy phosphatidylcholine, hydrogenated soy phosphatidylglycerol, dilauryloylphosphatidylcholine, dipalmitoylphosphatidylcholine, dioleoylphosphatidylycholine, dimyristoylphosphatidylethanolamine, dipalmitoylphosphatidylethanolamine, ioleoylphosphatidylethanolamine, dilauryloylphosphatidylglycerol, dipalmitoylphosphatidylglycerol, distearoylphosphatidylglycerol, dioleoylphosphatidylglycerol, dimyristoylphosphatidic acid, dioleoylphosphatidic acid, dipalmitoylphosphatidic acid, dimyristoylphosphatidylserine, dipalmitoylphosphatidylserine, and dioleoylphosphatidylserine. Preferably, two phospholipids are used in the method of this invention, one forming electronically neutral molecules in water (e.g., dimyristoylphosphatidylcholine) and the other forming electronically negative molecules in water (e.g., dimyristoylphosphatidylglycerol). The term “dispersing” refers to distributing a drug or phospholipids evenly throughout a medium, e.g., dissolving a water-soluble drug or suspending a water-insoluble drug in water.

To grind a mixture containing a drug and phospholipids, one can use any suitable mechanical means that reduces particle sizes. For example, one can add an aqueous mixture containing a drug and phospholipids in a mortar and grind the mixture with a pestle. Another example of a suitable mechanical means is a dispersion mill, e.g., an attritor mill, a vibrator mill, a media mill, or a ball mill.

To prepare a drug-lipid complex, the molar ratio between the drug and the lipids ranges from 1:9 to 9:1 (e.g., from 1:3 to 3:1 or from 2:3 to 3:2). The particle size of a drug-lipid complex thus obtained ranges from 60-6,000 nm (e.g., 250-3,000 nm or 400-1,200 nm). On the other hand, to prepare a drug-containing liposome, the molar ratio between the drug and the lipids ranges from 1:99 to 1:9 (e.g., from 1:50 to 1:9 or from 1:20 to 1:9). The particle size of a drug-containing liposome thus obtained ranges from 60-1,500 nm (e.g., 60-600 nm or 60-120 nm). The particle size mentioned herein refers to a number average particle size, as measured by conventional methods known in the art, such as photon correlation spectroscopy. Further, a stabilizer may be used to facilitate formation of drug-containing liposomes. Examples of a stabilizer include, but are not limited to, sterols (e.g., cholesterol) or tocopherols (e.g., DL-α-tocopherol).

When phosphatidylcholines (e.g., egg phosphatidylcholine or dimyristoylphosphatidylcholine) and phosphatidylglycerols (e.g., egg phosphatidylglycerol or dimyristoylphosphatidylglycerol) are used to practice the method of this invention, it is preferred that the molar ratio between phosphatidylcholines and phosphatidylglycerols range from 2:1 to 4:1.

The details of one or more embodiments of the invention are set forth in the description below. Other features, objects, and advantages of the invention will be apparent from the description and from the claims.

### DETAILED DESCRIPTION

This invention relates to an organic solvent-free method for preparing a drug-lipid complex that does not have a captured volume or a drug-containing liposome in the manner described in the summary section.

For example, one can add an aqueous mixture containing a drug (either water-soluble or water-insoluble) and two phospholipids in a mortar and subsequently grind the mixture using a pestle until all water evaporates to form a film. The film can then be hydrated to form a solution of a drug-containing liposome or a drug-lipid complex that does not have a captured volume. The process can be carried out repeatedly until a desired particle size range is obtained. Alternatively, two phospholipids can first be suspended in water and then ground to form a film. The film is subsequently hydrated by an aqueous solution containing a drug. The mixture thus formed is further ground repeatedly until a desired particle size range is obtained. The drug and phospholipids can also be added in sequences different from those mentioned above. In one example, to prepare a drug-containing liposome, a water-soluble drug can be added after the liposome has formed. In another example, to prepare a drug-lipid complex that does not have a captured volume, a drug (either water-soluble or water-insoluble) and a phospholipid can be dispersed in water and ground before another phospholipid is added. The particle sizes can be measured by taking a sample from the mixture during the grinding or after the grinding is completed.

As another example, the grinding can be performed by using a commercially available ball mill. In this method, one can add an aqueous mixture containing a drug (either water-solube or water-insoluble) and a phospholipid to the premix chamber of a ball mill and grind the mixture in the presence of a grinding media by circulating the drug-lipid mixture through the grinding chamber and the premix chamber. Another phospholipid can then be added during the grinding of the drug-lipid mixture. The mixture is then further ground until a desired particle size range is obtained. Alternatively, a drug and two phospholipids can be dispersed in water and then ground in the ball mill until a desired particle size range is obtained. The grinding media used in the ball mill can be spherical and can have an average size of 0.3-0.4 mm. The grinding can be carried out under the ambient temperature and pressure. However, it is preferred that the ball mill be cooled to minimize temperature elevation during the grinding, thereby avoiding degradation of the drug. The overall grinding time can vary widely depending on the process conditions selected.

One or more stabilizers (e.g., cholesterol or DL-α-tocopherol) may be added before or during grinding a drug-lipid mixture to facilitate formation of a drug-containing liposome. Further, other agents can also be added before or during the grinding of the drug-lipid mixture. For example, polyethylene glycol may be added to prolong the circulation time in the blood of a drug-containing liposome or a drug-lipid complex that does not have a captured volume.

The structure of the above-described drug-lipid complex or liposome can be characterized by suitable analytical methods, such as differential scanning calorimetry (Chapman, D., in Liposome Technology, Gregoriadis, G., ed., 1984, CRC Press, Boca Raton), ³¹P-NMR spectroscopy (Cullis et al., in Membrane Fluidity in Biology, 1982, Academic Press, Inc., London & New York), x-ray diffraction (Shipley et al., in Biomembranes, 1973, Chapman, D. and Wallach, D., eds., Vol. 2:1, Academic Press, Inc., London & New York), captured volume measurement (Deamer et al., Chem. Phys. Lipids, 1986, 40:167-188), and ultra-violet spectroscopy (Janoffet al., U.S. Pat. No. 6,406,713).

To practice the method of this invention, one uses a higher drug-lipid ratio (i.e., in the range of 1:9 to 9:1) to prepare a drug-lipid complex that does not have a captured volume. In contrast, one uses a lower drug-lipid ratio (i.e., in the range of 1:99 to 1:9) to prepare a drug-containing liposome. Typically, the drug-lipid complex that does not have a captured volume has a larger particle size (i.e., up to 6,000 nm) than that (i.e., up to 1,500 nm) of the drug-containing liposome.

The specific examples below are to be construed as merely illustrative, and not limitative of the remainder of the disclosure in any way whatsoever. Without further elaboration, it is believed that one skilled in the art can, based on the description herein, utilize the present invention to its fullest extent. All publications cited herein are hereby incorporated by reference in their entirety.

### EXAMPLE 1

### Preparation of a Doxorubicin-Containing Liposome Solution

520.1 mg of egg phosphatidylcholine (egg PC), 214.6 mg of egg phosphatidylglycerol (egg PG), 297.8 mg of cholesterol, and 3 mL of injectable water were added to an agate mortar. The mixture was wet ground using an agate pestle until all water evaporated, which took about 2 hours. The lipids were then mixed with additional water and ground continuously for 60 minutes. After the water evaporated, a dry lipid film formed on the surface of the agate mortar. A liposomal solution was then obtained by hydrating the lipid film with a 20 mL aqueous solution containing 3.6 mg/mL doxorubicin HCl (a water-soluble drug), 140 mM NaCl, 10 mM Tris-HCl, pH 4-5 at 30° C. for 30 minutes. The particle size range of the doxorubicin-containing liposome was measured using a Beckman Coulter N4 Plus Particle Size Analyzer (Beckman Coulter Inc., Fullerton, Calif.) and was determined to be 150-200 nm.

### EXAMPLE 2

### Preparation of a Taxol-Containing Liposome Solution

121.6 mg of egg PC, 6.1 mg of PEG 5000, 24.3 mg of cholesterol, 22.9 mg of DL-α-tocopherol, and 0.5 mL of water were mixed in an agate mortar. The mixture was ground by an agate pestle until all water evaporated. 3.6 mg of taxol (a water-insoluble drug) was then added to the mixture. It was then repeatedly mixed with 0.5 mL of water and ground in a manner similar to that described in Example 1 until the overall grinding time reached 120 minutes. After the water evaporated, a cake of a drug-lipid mixture formed on the surface of the agate mortar. A solution of a taxol-containing lipsome was obtained by hydrating the cake with 20 mL of a saline solution at 40° C. for 30 minutes. The particle size range of the taxol-containing liposome was determined to be 450-600 mn.

### EXAMPLE 3

### Preparation of an Amphotericin B-Containing Liposome Solution

20.4 mg of amphotericin B (AmB; a water-insoluble drug), 140.8 mg of DMPC, 61.4 mg of DMPG, and 0.5 mL of water were mixed in an agate mortar. The mixture was ground using an agate pestle until all water evaporated. It was then repeatedly mixed with 0.5 mL of water and ground in a manner similar to that described in Example 1 until the overall grinding time reached 120 minutes. After all water evaporated, a cake of a drug-lipid mixture formed on the surface of the agate mortar. A solution of an AmB-containing liposome was obtained by hydrating the cake with 20 mL pure water. The solution was then put in a bath, sonicated for 30 seconds, and stored at 2-8° C. The particle size range was determined to be 300-500 nm.

### EXAMPLE 4

### Preparation of an Amphotericin B-Lipid Complex Solution with High Drug/Lipids Ratio

200.9 mg of AmB, 137.3 mg of DMPC, 61.9 mg of DMPG, and 0.5 mL of water were mixed in an agate mortar. The mixture was ground using an agate pestle until all water evaporated. It was then repeatedly mixed with 0.5 mL of water and ground in a manner similar to that described in Example 1 until the overall grinding time reached 120 minutes. After all water evaporated, a cake of a drug-lipid mixture formed on the surface of the agate mortar. A solution of an AmB-lipid complex was obtained by hydrating the cake with 20 mL pure water. The solution was then put in a bath, sonicated for 30 seconds, and stored at 2-8° C. The particle size range was determined to be 2,000-3,000 nm.

Single dose acute toxicity study was performed using female ICR mice (National Taiwan University Laboratory Animal Center, Taipei, Taiwan). The mice were aged 5-6 weeks and weighed between 20-25 g. The observation was conducted in a period of 14 days.

An acute toxicity study was conducted for the AmB-lipid complex solution prepared above and FUNGIZONE, a free form of AmB. In particular, twenty female ICR mice were randomly and evenly divided into five groups. FUNGIZONE or the AmB-lipid complex solution of a predetermined dosage was intravenously injected to the mice in each group. The results indicated that the AmB-lipid complex had much lower toxicity level than that of FUNGIZONE. Specifically, the AmB-lipid complex had a LD₅₀ value (i.e., the lethal dose of the composition that kills half of the mice in a test group) greater than 80 mg/kg, while FUNGIZONE had a LD₅₀ value of about 5 mg/kg.

### EXAMPLE 5

### Preparation of an Amphotericin B-Lipid Complex Solution Using a Ball Mill

8.02 g of AmB, 2.65 g of DMPG, and 1000 g of a saline solution (containing 0.9% NaCl) were added to the premix chamber of a ball mill (Netzsch Inc., Exton, Pa.). The solution was stirred for about 10 minutes before ground by circulating through a grinding chamber and the premix chamber. After the AmB-DMPG suspension was circulated for 30 minutes, 5.48 g DMPC was introduced into the premix chamber and the mixture was circulated for another 180 minutes to obtain an AmB-lipid complex. The overall grinding time was 210 min. The operating conditions are listed as follows: (1) the grinding chamber was made polyamide; (2) the grinding vessel had a volume of 0.17 L; (3) the grinding media was glass beads (0.3-0.4 mm); (4) the pump was a hose pump (px 10 type); (5) the operating temperature was 20° C.; and (6) the agitator speed was 2600 rpm (12.3 m/s).

Samples were taken at a grinding time of 90 minutes, 150 minutes, and 210 minutes, and were designated as LDAB₉₀, LDAB₁₅₀, and LDAB₂₁₀, respectively. All samples were stored at 2-8° C. before analyzed. The particle size ranges of the samples were measured using N4 Plus Particle Size Analyzer and the AmB content was measured using high performance liquid chromatography. The results showed that AmB remained stable during the whole period of the grinding process and that the particle size reduced with an increasing grinding time.

Single dose acute toxicity study was performed using female BALB/c mouse (National Taiwan University Laboratory Animal Center, Taipei, Taiwan). Twelve female BALB/c mice aged 8-9 weeks and weighed 16-20 g were randomly and evenly divided into three groups. LDAB₉₀, LDAB₁₅₀, and LDAB₂₁₀ prepared above were intravenously injected to the mice in each group. The results showed toxicity reduced with an increasing grinding time and that at least 210 minutes was required under the condition described above to reach tolerable safety level in this animal model.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present invention, and without departing from the spirit and scope thereof, can make various changes and modifications of the invention to adapt it to various usages and conditions. Thus, other embodiments are also within the scope of the following claims.

1. A method for preparing a drug-lipid complex, comprising
dispersing a drug and one or more phospholipids in an aqueous solution to obtain a mixture, in which the molar ratio between the drug and the lipids ranges from 1:9 to 9:1; and
grinding the mixture with a mechanic means to obtain a drug-lipid complex that does not have a captured volume.

2. The method of claim 1, wherein the phospholipids are dimyristoylphosphatidylcholine and dimyristoylphosphatidylglycerol.

3. The method of claim 2, wherein the molar ratio between the drug and the lipids ranges from 1:3 to 3:1.

4. The method of claim 3, wherein the molar ratio between the drug and the lipids ranges from 2:3 to 3:2.

5. The method of claim 4, wherein the drug-lipid complex has a particle size of 60-6,000 nm.

6. The method of claim 5, wherein the drug-lipid complex has a particle size of 250-3,000 nm.

7. The method of claim 5, wherein the drug has a water solubility less than 10 mg/mL.

8. The method of claim 7, wherein the drug is amphotericin B, doxorubicin, taxol, or irinotecan.

9. The method of claim 8, wherein the molar ratio between dimyristoylphosphatidylcholine and dimyristoylphosphatidylglycerol ranges from 4:1 to 2:1.

10. The method of claim 1, wherein the molar ratio between the drug and the lipids ranges from 1:3 to 3:1.

11. The method of claim 10, wherein the molar ratio between the drug and the lipids ranges from 2:3 to 3:2.

12. The method of claim 1, wherein the drug-lipid complex has a particle size of 60-6,000 nm.

13. The method of claim 12, wherein the drug-lipid complex has a particle size of 250-3,000 nm.

14. The method of claim 1, wherein the drug has a water solubility less than 10 mg/mL.

15. The method of claim 14, wherein the drug is amphotericin B, doxorubicin, taxol, or irinotecan.

16. The method of claim 1, wherein the mechanic means is a dispersion mill.

17. The method of claim 16, wherein the dispersion mill is a ball mill.

18. The method of claim 17, wherein the phospholipids are dimyristoylphosphatidylcholine and dimyristoylphosphatidylglycerol.

19. The method of claim 18, wherein the molar ratio between the drug and the lipids ranges from 1:3 to 3:1.

20. The method of claim 19, wherein the molar ratio between the drug and the lipids ranges from 2:3 to 3:2.

21. The method of claim 20, wherein the drug-lipid complex has a particle size of 60-6,000 nm.

22. The method of claim 21, wherein the drug-lipid complex has a particle size of 250-3,000 nm.

23. The method of claim 21, wherein the drug has a water solubility less than 10 mg/mL.

24. The method of claim 23, wherein the drug is amphotericin B, doxorubicin, taxol, or irinotecan.

25. The method of claim 24, wherein the molar ratio between dimyristoylphosphatidylcholine and dimyristoylphosphatidylglycerol ranges from 4:1 to 2:1.

26. The method of claim 16, wherein the molar ratio between the drug and the lipids ranges from 1:3 to 3:1.

27. The method of claim 26, wherein the molar ratio between the drug and the lipids ranges from 2:3 to 3:2.

28. The method of claim 16, wherein the drug-lipid complex has a particle size of 60-6,000 nm.

29. The method of claim 28, wherein the drug-lipid complex has a particle size of 250-3,000 nm.

30. The method of claim 16, wherein the drug has a water solubility less than 10 mg/mL.

31. The method of claim 30, wherein the drug is amphotericin B, doxorubicin, taxol, or irinotecan.

32. A method for preparing a drug-containing liposome, comprising
dispersing a drug and one or more phospholipids in an aqueous solution to obtain a mixture, in which the molar ratio between the drug and the lipids ranges from 1:99 to 1:9; and
grinding the mixture with a mechanic means to obtain a drug-containing liposome.

33. The method of claim 32, wherein the phospholipids are egg phosphatidylcholine and egg phophatidylglycerol.

34. The method of claim 33, further comprises dispersing a stabilizer in the aqueous solution.

35. The method of claim 34, wherein the drug-containing liposome has a particle size of 60-1,500 nm.

36. The method of claim 35, wherein the drug-containing liposome has a particle size of 60-600 nm.

37. The method of claim 35, wherein the drug has a water solubility less than 10 mg/mL.

38. The method of claim 37, wherein the drug is amphotericin B, doxorubicin, taxol, or irinotecan.

39. The method of claim 38, wherein the molar ratio between egg phosphatidylcholine and egg phosphatidylglycerol ranges from 4:1 to 2:1.

40. The method of claim 32, wherein the drug-containing liposome has a particle size of 60-1,500 nm.

41. The method of claim 40, wherein the drug-containing liposome has a particle size of 60-600 nm.

42. The method of claim 32, wherein the drug has a water solubility less than 10 mg/mL.

43. The method of claim 42, wherein the drug is amphotericin B, doxorubicin, taxol, or irinotecan.

44. The method of claim 32, wherein the mechanic means is a dispersion mill.

45. The method of claim 44, wherein the dispersion mill is a ball mill.

46. The method of claim 45, wherein the phospholipids are egg phosphatidylcholine and egg phophatidylglycerol.

47. The method of claim 46, further comprises dispersing a stabilizer in the aqueous solution.

48. The method of claim 47, wherein the drug-containing liposome has a particle size of 60-1,500 nm.

49. The method of claim 48, wherein the drug-containing liposome has a particle size of 60-600 nm.

50. The method of claim 48, wherein the drug has a water solubility less than 10 mg/mL.

51. The method of claim 50, wherein the drug is amphotericin B, doxorubicin, taxol, or irinotecan.

52. The method of claim 51, wherein the molar ratio between egg phosphatidylcholine and egg phosphatidylglycerol ranges from 4:1 to 2:1.

53. The method of claim 44, wherein the drug-containing liposome has a particle size of 60-1,500 nm.

54. The method of claim 53, wherein the drug-containing liposome has a particle size of 60-600 nm.

55. The method of claim 44, wherein the drug has a water solubility less than 10 mg/mL.

56. The method of claim 55, wherein the drug is amphotericin B, doxorubicin, taxol, or irinotecan.
